(19) United States
(12) Patent Application Publication
(10) Pub. No.: US 2025/0026643 A1
(43) Pub. Date: Jan. 23, 2025

(54) PRODUCTION OF ANHYDROUS QUATERNARY AMMONIUM HYDROXIDES

(71) Applicant: Huntsman Petrochemical LLC, The Woodlands, TX (US)

(72) Inventors: Hui ZHOU, The Woodlands, TX (US); Ke ZHANG, The Woodlands, TX (US)

(21) Appl. No.: 18/712,736

(22) PCT Filed: Dec. 2, 2022
(86) PCT No.: PCT/US2022/051625

§ 371 Date: May 23, 2024

Related U.S. Application Data
(60) Provisional application No. 63/285,767, filed on Dec. 3, 2021.

(51) Int. Cl. C01B 21/087

(52) U.S. Cl. CPC C01B 21/087

(57) ABSTRACT

A method for the synthesis of anhydrous quaternary ammonium hydroxides having the formula (NR₁R₂R₃R₄) OH, wherein R₁, R₂, R₃ and R₄ are the same as or different from one another and are each individually selected from H, C₁-C₁₀ alkyls including linear or cyclo alkyls, and C₆-C₁₂ aryls by reacting an alkylene oxide with a tertiary amine having the formula R₁R₂R₃N wherein R₁, R₂, and R₃ are the same or different and are each individually selected from C₁-C₁₀ alkyls including linear and cyclo alkyls, C₁-C₁₀ hydroxyalkyls, C₆-C₁₂ aryls, and formulations having anhydrous quaternary ammonium hydroxides.

### CROSS REFERENCE TO RELATED APPLICATIONS

This application claims priority to U.S. Provisional Patent Application Ser. No. 63/285,767 filed Dec. 3, 2021. The noted application is incorporated herein by reference.

### FIELD

The present disclosure generally relates to a method for the production of anhydrous quaternary ammonium hydroxides. More specifically, the present disclosure relates to the synthesis of anhydrous quaternary ammonium hydroxides by the addition reaction of alkylene oxide to tertiary amines for electronic applications, such as cleaning, stripping, or etching formulations.

### BACKGROUND

In the electronics industry aqueous quaternary ammonium compounds, such as, tetramethylammonium hydroxide (TMAH), choline hydroxide, etc., have been widely used in the formulations for etching, stripping, cleaning process to develop metallic or semiconductor architecture. Aqueous formulations can cause watermarks (drying spots) to form during the drying process. Solvent based formulations have been reported and used to reduce watermarks, however, such formulations can be limiting due to solvent incompatibility. Unlike water- or solvent-based quaternary ammonium hydroxide, which limits its application to either aqueous solutions or specific solvent-based solutions, anhydrous quaternary ammonium hydroxide can provide users with more options for their formulations. Such anhydrous quaternary ammonium hydroxides can be formulated in either water- or solvent-based applications.

Despite the state of the art, there is a continuous need for the development of methods to synthesize anhydrous quaternary ammonium hydroxides for use in electrical applications.

### DETAILED DESCRIPTION

Before explaining aspects of the present disclosure in detail, it is to be understood that the present disclosure is not limited in its application to the details of construction and the arrangement of components or steps or methodologies set forth in the following description. The present disclosure is capable of other embodiments or of being practiced or carried out in various ways. Also, it is to be understood that the phraseology and terminology employed herein is for the purpose of description and should not be regarded as limiting.

Unless otherwise defined herein, technical terms used in connection with the present disclosure shall have the meanings that are commonly understood by those having ordinary skill in the art. Further, unless otherwise required by context, singular terms shall include pluralities and plural terms shall include the singular.

All patents, published patent applications, and non-patent publications mentioned in the specification are indicative of the level of skill of those skilled in the art to which the present disclosure pertains. All patents, published patent applications, and non-patent publications referenced in any portion of this application are herein expressly incorporated by reference in their entirety to the same extent as if each individual patent or publication was specifically and individually indicated to be incorporated by reference to the extent that they do not contradict the instant disclosure.

All of the compositions and/or methods disclosed herein can be made and executed without undue experimentation in light of the present disclosure. While the compositions and methods of the present disclosure have been described in terms of preferred embodiments, it will be apparent to those having ordinary skill in the art that variations may be applied to the compositions and/or methods and in the steps or sequences of steps of the methods described herein without departing from the concept, spirit, and scope of the present disclosure. All such similar substitutes and modifications apparent to those skilled in the art are deemed to be within the spirit, scope, and concept of the present disclosure.

As utilized in accordance with the present disclosure, the following terms, unless otherwise indicated, shall be understood to have the following meanings.

The use of the word “a” or “an”, when used in conjunction with the term “comprising”, “including”, “having”, or “containing” (or variations of such terms) may mean “one”, but it is also consistent with the meaning of “one or more”, “at least one”, and “one or more than one”.

The use of the term “or” is used to mean “and/or” unless clearly indicated to refer solely to alternatives and only if the alternatives are mutually exclusive.

If the specification states a component or feature “may,” “can,” “could,” or “might” be included or have a characteristic, that particular component or feature is not required to be included or have the characteristic.

Throughout this disclosure, the term “about” is used to indicate that a value includes the inherent variation of error for the quantifying device, mechanism, or method, or the inherent variation that exists among the subject(s) to be measured. For example, but not by way of limitation, when the term “about” is used, the designated value to which it refers may vary by plus or minus ten percent, or nine percent, or eight percent, or seven percent, or six percent, or five percent, or four percent, or three percent, or two percent, or one percent, or one or more fractions therebetween.

The use of “at least one” will be understood to include one as well as any quantity more than one, including but not limited to, 1, 2, 3, 4, 5, 10, 15, 20, 30, 40, 50, 100, etc. The term “at least one” may extend up to 100 or 1000 or more depending on the term to which it refers. In addition, the quantities of 100/1000 are not to be considered as limiting since lower or higher limits may also produce satisfactory results.

In addition, the phrase “at least one of X, Y, and Z” will be understood to include X alone, Y alone, and Z alone, as well as any combination of X, Y, and Z. Likewise, the phrase “at least one of X and Y” will be understood to include X alone, Y alone, as well as any combination of X and Y. Additionally, it is to be understood that the phrase “at least one of” can be used with any number of components and have the similar meanings as set forth above.

As used herein, the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The phrases “in one example”, “in an example”, “according to one example”, and the like generally mean the particular feature, structure, or characteristic following the phrase is included in at least one example of the present disclosure, and may be included in more than one example of the present disclosure. Importantly, such phrases are non-limiting and do not necessarily refer to the same example but, of course, can refer to one or more preceding and/or succeeding examples. For example, in the appended claims, any of the claimed examples can be used in any combination.

As used herein, the terms “% by weight”, “wt %”, “weight percentage”, or “percentage by weight” are used interchangeably.

As used herein, the term “ambient temperature” refers to the temperature of the surrounding work environment (e.g., the temperature of the area, building or room where the curable composition is used), exclusive of any temperature changes that occur as a result of the direct application of heat to the curable composition to facilitate curing. The ambient temperature is typically between about 10° C. and about 30° C., more specifically about 15° C. and about 25° C.

Described herein is the preparation of an anhydrous quaternary ammonium hydroxide which can be used in either water- or solvent-based applications. The processes described herein can be used to prepare an anhydrous quaternary ammonium hydroxide compound having the formula (NR₁R₂R₃R₄) OH, wherein R₁, R₂, R₃ and R₄ are the same as or different from one another and are each individually selected from H, C₁-C₁₀ alkyls including linear or cyclo alkyls, C₁-C₁₀ hydroxylalkyls, and C₆-C₁₂ aryls.

Specifically, the processes described herein can include reacting an alkylene oxide with a tertiary amine having the formula R₁R₂R₃N, wherein R₁, R₂ and R₃ are the same as or different from one another and are each individually selected from C₁-C₁₀ alkyls including linear or cyclo alkyls, C₁-C₁₀ hydroxyalkyls, and C₆-C₁₂ aryls, to yield the anhydrous quaternary ammonium hydroxide. In at least one example, the reaction of alkylene oxide and tertiary amine can be performed in an aqueous environment. In an alternative example, the reaction can be performed in an anhydrous environment using one or more solvents including, without limitation, N-methyl-2-pyrrolidone, dimethyl sulfoxide, dimethylacetamide, t-butyl alcohol, ethylene glycol, and propylene glycol.

Alkylene oxides which can be used to produce the presently disclosed anhydrous quaternary ammonium hydroxide can include, without limitation, ethylene oxide, propylene oxide, butylene oxide, allyl glycidyl ether, hexene oxide, styrene oxide, epichlorohydrin, cyclohexene oxide, spiro-epoxy oxindole, and the like.

Tertiary amines which can be used to produce the presently disclosed anhydrous quaternary ammonium hydroxide can include, without limitation, trimethylamine, triethylamine, tripropylamine, tributlylamine, triocylamine, dimethylethylamine, diethylmethylamine, dimethylbenzylamine, dimethylaniline, dimethylcyclohexylamine, diethylbenzylamine, dimethylethanolamine, methyldiethanolamine, triethanolamine, dimethylpropanolamine, diethylethanolamine, ethyldiethanolamine, and methylethylpropanolamine.

Byproducts of the presently process can include water and unreacted amine. The amount of water generated as a byproduct of the reaction can be maintained at extremely low levels. In at least one example, the water generated as a byproduct can be controlled at less than about 5% by weight. In an additional example, the water generated as a byproduct can be controlled at less than about 1% by weight.

Generally, the molar ratio of alkylene oxide to tertiary amine present in the reaction described herein ranges from about 6:1 to about 1:3, preferably from about 4:1 to about 1:2, and more preferably from about 2:1 to about 1:1.

The reaction can be performed at a temperature range of from about ambient temperature to about 70° C. In at least one example, the reaction temperature can range from about 30° C. to about 60° C. The reaction can be performed at a pressure of less than about 60 psi. In at least one example, the reaction pressure can be less than about 55 psi.

The quaternary ammonium hydroxides produced by the methods described herein can provide a better EHS profile including, without limitation, lower toxicity as compared to those conventionally made using tetramethylammonium hydroxide (TMAH) or choline hydroxide. For example, TMAH is commonly used at a 2.38% by weight aqueous solution, which has high levels of toxic materials.

Formulation requirements for etching and cleaning different metals, photoresist, organics can vary for analog, logic, dynamic random access memory (DRAM), NAND semiconductor devices, as well as display and photovoltaic devices can vary drastically. The concentration of the starting materials can be adjusted to achieve a desired strength of the resulting anhydrous quaternary ammonium hydroxide. In at least one example, changing the tertiary amines precursor structure can allow the anhydrous quaternary ammonium hydroxide etching capability and hydrophilicity to be tailored for specific end uses. The processes can therefore enable the formation of a variety of anhydrous quaternary ammonium hydroxide formulas for various uses including, without limitation, etching, stripping, and cleaning purposes. The final formulations can be either water- or solvent-based.

The methods presently disclosed provides an anhydrous quaternary ammonium hydroxide which can have increased freedom in formulation design for a specific wet processing step, such as a specific type or mixture of metal/semiconductor surfaces, nature of prior processing residues, or a certain effect on a surface is desired. A final formulation can be created with water or a solvent. In at least one example, solvent can be an isopropyl alcohol or an ethylene oxide reactive solvent.

Examples of the present synthesis reaction are provided below. However, the present disclosure is to be understood to not be limited in its application to the specific experiments, results, and laboratory procedures disclosed herein below. Rather, the Examples are simply provided as one of various embodiments and are meant to be exemplary and not exhaustive.

### EXAMPLES

### Example 1

In a clean and dry 8-gallon kettle equipped with an agitator and nitrogen line, 6.33 pounds (lbs) methyldiethanolamine and 0.53 lb water were charged and padded with nitrogen. With the cooling on, ethylene oxide was introduced at ambient temperature and pressure. The temperature was maintained at 30° C. The addition rate of ethylene oxide must be low initially, but can be ramped up gradually in order to maintain the reaction temperature. After 1.25 lbs of ethylene oxide was added, the digestion was continued for 1 hour at 50 psi max. The kettle can be slowly vented while nitrogen can be used to strip the residual ethylene oxide to the scrubber for 1 hour. Under minimized air contact, 0.064 lb of stabilizer was added under nitrogen. After fully mixed, the final product was discharged into a plastic container.

Titration of the final product indicated about 42% by weight quaternary ammonium hydroxides, 2% by weight free water and excess methyldiethanolamine. The pH of the final product was determined to be about 12.9.

### Example 2

In a clean and dry 8-gallon kettle equipped with an agitator and nitrogen line, 6.37 lbs methyldiethanolamine and 0.71 lb deionized (DI) water were charged and padded with nitrogen. With the cooling on, ethylene oxide was introduced at ambient temperature and pressure. The temperature was maintained at 30° C. The addition rate of ethylene oxide must be low initially, but can be ramped up gradually in order to maintain the reaction temperature. After 1.74 lbs of ethylene oxide was added, the digestion was continued for 1 hour at 50 psi max. The kettle can be slowly vented while nitrogen can be used to strip the residual ethylene oxide to the scrubber for 1 hour. Under minimized air contact, 0.070 lb of stabilizer was added under nitrogen. After fully mixed, the final product was discharged into a plastic container.

Titration of the final product indicated about 54% by weight quaternary ammonium hydroxides, 3% by weight free water and excess methyldiethanolamine. The pH of the final product was determined to be about 13.

### Example 3

In a clean and dry 8-gallon kettle equipped with an agitator and nitrogen line, 6.33 lbs methyldiethanolamine and 0.53 lb deionized (DI) water were charged and padded with nitrogen. With the cooling on, ethylene oxide was introduced at ambient temperature and pressure. The temperature was maintained at 30° C. The addition rate of ethylene oxide must be low initially, but can be ramped up gradually in order to maintain the reaction temperature. After 2.51 lbs of ethylene oxide was added, the digestion was continued for 1 hour at 50 psi max. The kettle can be slowly vented while nitrogen can be used to strip the residual ethylene oxide to the scrubber for 1 hour. Under minimized air contact, 0.064 lb of stabilizer was added under nitrogen. After fully mixed, the final product was discharged into a plastic container.

Titration of the final product indicated about 69% by weight quaternary ammonium hydroxides and excess methyldiethanolamine. No free water was detected. The pH of the final product was determined to be about 13. The product is very high viscosity, paste like liquid.

From the above description, it is clear that the present disclosure is well adapted to carry out the object and to attain the advantages mentioned herein as well as those inherent in the present disclosure. While exemplary embodiments of the present disclosure have been described for the purposes of the disclosure, it will be understood that numerous changes may be made which will readily suggest themselves to those skilled in the art which can be accomplished without departing from the scope of the present disclosure and the appended claims.

1. A method for the synthesis of an anhydrous quaternary ammonium hydroxide comprising:
reacting an alkylene oxide with a tertiary amine having the formula R1R2R3N wherein R1, R2, and R3 are the same or different and are each individually selected from C₁-C₁₀ alkyls, C₁-C₁₀ hydroxyalkyls and C₆-C₁₂ aryls, to produce an anhydrous quaternary ammonium hydroxide having the formula (NR₁R₂R₃R₄) OH, wherein R₁, R₂, R₃, and R₄ are the same as or different from one another and are each individually selected from H, C₁-C₁₀ alkyls, C₁-C₁₀ hydroxyalkyls and C₆-C₁₂ aryls.

2. The method of claim 1, wherein the alkylene oxide is selected from ethylene oxide, propylene oxide, butylene oxide, allyl glycidyl ether, hexene oxide, styrene oxide, epichlorohydrin, cyclohexene oxide and spiro-epoxy oxindole.

3. The method of claim 1, wherein the tertiary amine is selected from trimethylamine, triethylamine, tripropylamine, dimethylethylamine, diethylmethylamine, dimethylethanolamine, methyldiethanolamine, triethanolamine, dimethylpropanolamine, diethylethanolamine and methylethylpropanolamine.

4. The method of claim 1, wherein the reaction is performed in an aqueous environment.

5. The method of claim 1, wherein the reaction produces byproducts including water and unreacted amine.

6. The method of claim 5, wherein the reaction produces less than about 5% by weight water as byproduct.

7. An electronic application formulation comprising an anhydrous quaternary ammonium hydroxide as claimed in claim 1.

8. The electronic application formulation of claim 7, further comprising a solvent including isopropyl alcohol or an ethylene oxide reactive solvent.

9. The electronic application formulation of claim 7, further comprising water.

10. An anhydrous quaternary ammonium hydroxide composition obtained by reacting an alkylene oxide with a tertiary amine having the formula R1R2R3N wherein R1, R2, and R3 are the same or different and are each individually selected from C₁-C₁₀ alkyls, C₁-C₁₀ hydroxyalkyls and C₆-C₁₂ aryls.

11. The anhydrous quaternary ammonium hydroxide composition of claim 10, wherein the alkylene oxide is selected from ethylene oxide, propylene oxide, butylene oxide, allyl glycidyl ether, hexene oxide, styrene oxide, epichlorohydrin, cyclohexene oxide, and spiro-epoxy oxindole.

12. The anhydrous quaternary ammonium hydroxide composition of claim 11, wherein the alkylene oxide is ethylene oxide.

13. The anhydrous quaternary ammonium hydroxide composition of claim 10, wherein R1, R2 and R3 are individually selected from C₁-C₁₀ alkyls.

14. The anhydrous quaternary ammonium hydroxide composition of claim 13, wherein R1, R2 and R3 are individually selected from methyl and ethyl.

15. The anhydrous quaternary ammonium hydroxide composition of claim 10, wherein R1, R2 and R3 are individually selected from C₁-C₁₀ hydroxyalkyls.

16. The anhydrous quaternary ammonium hydroxide composition of claim 10, wherein a molar ratio of alkylene oxide to tertiary amine present in the reaction ranges from about 6:1 to about 1:3.

17. The anhydrous quaternary ammonium hydroxide composition of claim 10, wherein the reaction is performed in the presence of a solvent including N-methyl-2-pyrrolidone, dimethyl sulfoxide, dimethylacetamide, t-butyl alcohol, ethylene glycol, or propylene glycol.

18. The anhydrous quaternary ammonium hydroxide composition of claim 10, wherein the reaction is conducted at a temperature in the range from about 30° C. to about 60° C.

19. The anhydrous quaternary ammonium hydroxide composition of claim 18, wherein the reaction is conducted at a pressure less than about 60 psi.

20. A method for etching, stripping or cleaning a surface of a semiconductor device including contacting the surface with the electronic application formulation of claim 7.
